(19) United States
(12) Patent Application Publication
(10) Pub. No.: US 2013/0296645 A1
(43) Pub. Date: Nov. 7, 2013

(54) Male and Female Enhancing Device

(71) Applicant: Percy Evans, Coral Springs, FL (US)

(72) Inventor: Percy Evans, Coral Springs, FL (US)

(21) Appl. No.: 13/466,051

(22) Filed: May 7, 2012

(51) Int. Cl. A61F 5/41

(52) U.S. Cl. 600/41

(57) ABSTRACT

A male and female sexual enhancing device for enhancing a flaccid penis and providing sexual stimulation to a vagina. The sexual enhancing device permits for a minimal coverage of a penis surface area, while also allowing enhanced sexual stimulation for both the male and female through the utilization of a rigid, extended assembly that enhances the flaccid penis. The components that create the enhanced erection include a bulbous distal member for extending and widening the penis. Also, two annular members receive and support the penis and reduce blood flow to the penis. A longitudinal stiffening rod provides perpetual rigidity to the penis, and a plurality of convex stimulators positioned onto the erection enhancing device maximizes sexual stimulation. Vaginal stimulators such as clit ticklers and vibrators attach along the shaft base for stimulating the vagina. The sexual enhancing device is utilized by the male and the female together or alone.

### FIELD OF THE INVENTION

The present invention relates to a sexual enhancing device. More so, the sexual enhancing device allows for minimal coverage of a penis surface area, while also allowing enhanced sexual stimulation for both a male and a female through the utilization of a rigid, extended assembly that enhances a flaccid penis and numerous vaginal stimulating members.

### BACKGROUND OF THE INVENTION

A flaccid penis is a limp or soft penis. This is a desirable state for a penis to be in for most everyday activities. A rigid, protruding penis at work or at a social function is a cause of distraction and is also be considered gauche in most societies. However, during sexual activities, a flaccid penis is not a desirable condition.

During sexual encounters, the flaccid penis carries with it a negative stigma, often referred to as “penile dysfunction”, “impotence”, or in slang, “limp dick”. While the cause of the flaccid penis is most often a lack of blood flow, nervous jitters, stress, or simply old age, the psychological effects can be damaging—especially to the male. There is also physical fallout, as the female cannot not be obtain a full vaginal orgasm if the penis does not penetrate deeply into her sensitive regions; nor does the male receive his sexual gratification without the full penis shaft contacting the walls of the vagina. This conundrum has been studied for millennium, with a hodge podge of different solutions developed and tried over time. Not all have been efficacious in strengthening or lengthening the penis.

Medical herbs have been around since recorded history. The ancients have concocted myriad herbs and animal parts in their attempt to cure the flaccid penis. One of the most widely known herbs to help penis growth is Catuaba, an herb from the northern Amazon area in Brazil. Used for centuries by the local indigenous population, doctors in the area often prescribe it for erectile dysfunction. Another all-natural herb is From the Far East. Epimedium has been used to help penis growth and used in China and India for hundreds of years to enhance erectile disorders by increasing natural levels of testosterone. However, these herbs have recently been proven to have more of a psychological effects, rather than a physiological one. In modern times, large pharmaceutical companies have developed drugs such as Viagra and Cialis. These drugs are, for the most part, efficacious in treating erectile dysfunction. They work by blocking a chemical that causes blood to leave the penis. More blood in the penis means an improved erection. However, they have been linked to vision problems and causation of high blood pressure. Furthermore, these drugs are relatively expensive when used on a regular basis.

Annular-shaped members, often referred to as cock rings, prolong male erection by holding blood inside the penis. The increased blood flow prolongs period of the erection during sexual activity. A man may wear a cock-ring to combat erectile difficulties, or for the sensation of tightness and engorgement that wearing one provides. Some models include a protruding clitoral stimulator, designed to stimulate the clitoris during sex. Others vibrate, either vibrating the ring itself, or in a popular ‘dolphin’ variant by using two removable bullet vibrators to provide stimulation to both the testicles and clitoris. However, these devices do nothing to extend the length of the penis. The erect penis cannot suddenly increase longitudinally simple because it remains erect for longer periods of time.

A penis sleeve is a cylindrical device that is placed on the shaft of the penis, with the aim of increasing stimulation for the person being penetrated. The penis sleeve often has soft convex bumps intended to provide further stimulation. The penis sleeve can even include a bulbous distal member to increase the length of the penis. However, the penis sleeve envelopes the whole penis shaft, or most of it. And the penis sleeve is usually fabricated from materials such as rubber, thermoplastic elastomer, sheep skin, and the like. Thus, the male receives significantly less sensual stimulation than if the penis was fully exposed to the vaginal wall.

A penis extender is a partially hollow device like a very short dildo, with the hollow end placed on the end of the penis. The penis extender is intended to increase the effective-length of the penis, again for the benefit of the person being penetrated. The penis extender is generally worn with condoms to prevent them from falling off during use. While the penis extender does solve the problem of extending the penis, it fails in the same category as above namely, the male's penis shaft is fully covered, and thus does not receive full sensual contact with the vagina.

Even though the above cited penis stimulators and extenders address some of the needs of the market, an erection enhancing device that provides the flaccid penis with constant rigidity and sufficient length, while still allowing for significant exposure of the penis surface area is still desired. If such a device was disclosed, the male would be more confident and would receive increased sexual gratification during intercourse because of the significant exposure and perpetual rigidity of the penis. Similarly, the female would also receive enhanced sensual stimulation because of the increased length and width of the penis.

### SUMMARY OF THE INVENTION

This invention is directed to a sexual device or sexually stimulating both a male and a female simultaneously or alone. For the male, the sexual enhancing device may provide an erection enhancement that allows for significant exposure of a penis surface area while also providing a rigid and extended framework for perpetually erecting and enlarging a flaccid penis during sexual activity. The sexual enhancing device may also be fabricated from a skin soft material to provide maximum stimulation for both the male and female. The sexual enhancing device also comprises a bulbous distal member for extending and widening a penis, and a gonad supporting member to provide more support. The device utilizes a design that creates a vacuum around the penis for a tighter fit around the penis. For the female, the sexual enhancing device provides includes, in a strategic location, numerous vaginal stimulating members, such as, without limitation, a clit tickler, and a multi-speed vibrator. The erection enhancing device has many novel features. These features enhance the sensual affects of sexual activity. The erection enhancing device is configured to slidably position onto the penis and provides sensual gratification for both the male and female, even during rigrous sexual activity. The erection enhancing device provides a bulbous distal member abutting against the penis distal head that adds 1-4 inches of extension and an increased diameter to the penis. A chief novelty feature of the erection enhancing device is that up to 80% of the penis surface area remains uncovered and exposed while a user is wearing and using the erection enhancing device. Furthermore, annular members along the longitudinal shaft restrict blood flow to help prolong the erection, while a longitudinal stiffening rod positioned along the length of the penis provides rigidity and width to the penis shaft. A plurality of convex stimulators such as beads, flexible members, and ridges are located in strategic locations to provides sensual stimulation for the female. The erection enhancing device comprises a shaft base configured to slidably accept a penis shaft and fabricated from a rubber, thermoplastic elastomer, plastic, silicone, or the like. At least two annular members, often referred to as “cock rings” in commercial embodiments, attach to the shaft base. One embodiment includes a rear annular member that abuts against the lower abdomen and testicles, and a frontal annular member to secure the penis shaft to the shaft base. A longitudinal stiffening rod located inside the shaft base aligns along the full length of the shaft base and provides rigidity to the erection enhancing device, which indirectly stiffens the penis. A bulbous distal member attaches to the distal end of the shaft base and provides an extension for the penis. A plurality of convex stimulators is positioned along the shaft base, annular members, and bulbous distal member for providing enhanced sensual stimulation for the male and the female. Another embodiment of the erection enhancing device comprises at least one vaginal stimulating member such as a clit tickler and a multi-speed vibrator that attach to the erection enhancing device. The vaginal stimulating members cause enhanced agitation to the vagina and the penis, whereby the sensual stimulation for both the male and female is enhanced.

A first aspect of the present invention provides the male and female sexual enhancing device comprising:

a shaft base oriented in a longitudinal direction that is conductive for supporting a penis, the shaft base configured to slidably receive the penis;

a rear annular member attached to the shaft base, the rear annular member forming a perimeter around the penis shaft and abutting against the lower abdomen and testicles, wherein the rear annular member restricts blood flow in the penis and secures the penis to the erection enhancing device;

a frontal annular member attached to the shaft base, the frontal annular member forming a perimeter around the penis shaft, wherein the frontal annular member restricts blood flow in the penis and secures the penis to the erection enhancing device,

the longitudinal stiffening rod located inside the shaft base, the longitudinal stiffening rod further aligned longitudinally along the shaft base for providing rigidity and width to the penis shaft,

the bulbous distal member attached to an extreme end of the shaft base, the bulbous distal member abutting against the penis distal head, wherein the bulbous distal member provides length and width to the penis;

at least one convex stimulator attached to the erection enhancing device, wherein the convex stimulator is configured into a shape conducive for sexual stimulation; and

at least one vaginal stimulating member for agitating a vagina.

In a second aspect, the erection enhancing device allows up to 80% of the penis surface area to remain uncovered for maximizing sensual stimulation for the penis during sexual activity.

In another aspect, the longitudinal stiffening rod creates a rigid structure and increased width for the penis, even while the penis is flaccid.

In another aspect, additional annular member can be utilized with different embodiments of the erection enhancing device.

In another aspect, the convex stimulators are locate din a plurality of alignments on the erection enhancing device to maximize sensual stimulation for the female during sexual activity.

In another aspect, the convex stimulators are shaped as bumps, ridges, mushrooms, and similar designs that are efficacious in providing enhanced sexual stimulation for the female.

In another aspect, the bulbous distal member is fabricated form a flexible, rubber-like material such as silicone, thermoplastic elastomer, plastic, or material of similar extrusion.

In another aspect, a vibrating device attaches to the erection enhancing device, wherein the erection enhancing device is caused to vibrate.

In another aspect, in operation, the penis is stimulated into a 90 degree angle with the abdomen. If the penis is flaccid, the penis distal head is pulled out to the 90 degree position. Then, the erection enhancing device is oriented in front of the penis, so that the bulbous distal member is farthest from the lower abdomen. The erection enhancing device is then slidably pulled onto the penis until the rear annular member abuts against the lower abdomen and testicles and the bulbous distal member abuts against the penis distal head. Despite the erection enhancing device being fabricated in a manner that eliminates scratching or harming the penis, the erection enhancing device should still be pulled onto the penis slowly. The penis now rigid and extended, is then capable of being positioned inside a cavity for sexual activity.

In yet another aspect, the female can utilize the erection enhancing device without a penis due to the rigid structure of the device and the vaginal stimulating member such as ticklers and vibrators that attach to the device.

One benefit of the erection enhancing device is that it allows up to 80% of the penis surface area to remain uncovered and exposed for enhanced stimulation during sexual activity.

Another benefit of the erection enhancing device is that it provides increased dimensions for the penis, wherein the female receives enhanced sensual stimulation.

Another benefit of the erection enhancing device is that it is capable of creating a vibrating motion to enhance sexual stimulation for both the male and female simultaneously or alone.

Another benefit of the erection enhancing device is that it is lightweight and portable.

These and other advantages of the invention will be further understood and appreciated by those skilled in the art by reference to the following written specification, claims and appended drawings.

### BRIEF DESCRIPTION OF THE DRAWINGS

The invention will now be described, by way of example, with reference to the accompanying drawings, in which:

FIG. 1 presents an exemplary perspective side view of the erection enhancing device with a sectioned view of a longitudinal stiffening rod, in accordance with an embodiment of the present invention;

FIG. 2 presents an exemplary perspective front view of the erection enhancing device, in accordance with an embodiment of the present invention; and

FIG. 3 presents an exemplary elevated side view of the erection enhancing device with a penis inserted inside, positioned inside a vagina, in accordance with an embodiment of the present invention.

Like reference numerals refer to like parts throughout the various views of the drawings.

### DETAILED DESCRIPTION OF THE INVENTION

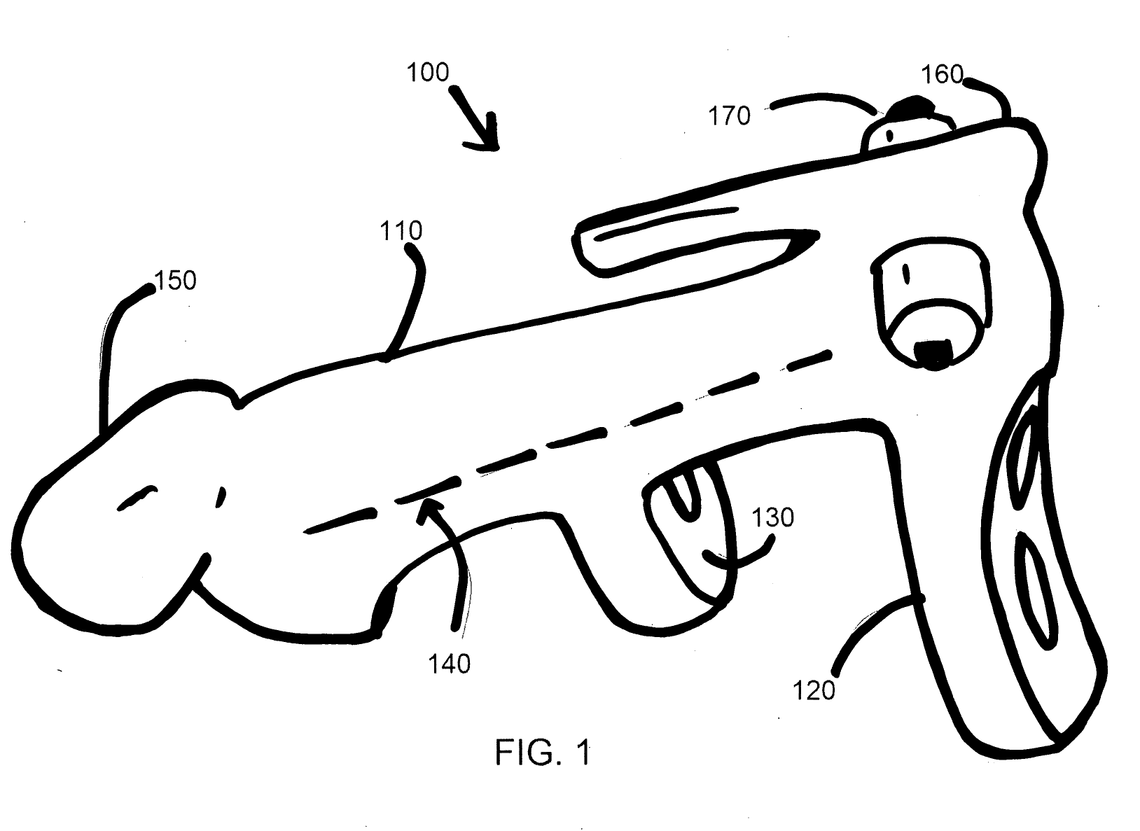

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations, All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “vertical,” “horizontal,” and derivatives thereof shall relate to the invention as oriented in FIGS. 1 through 3. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

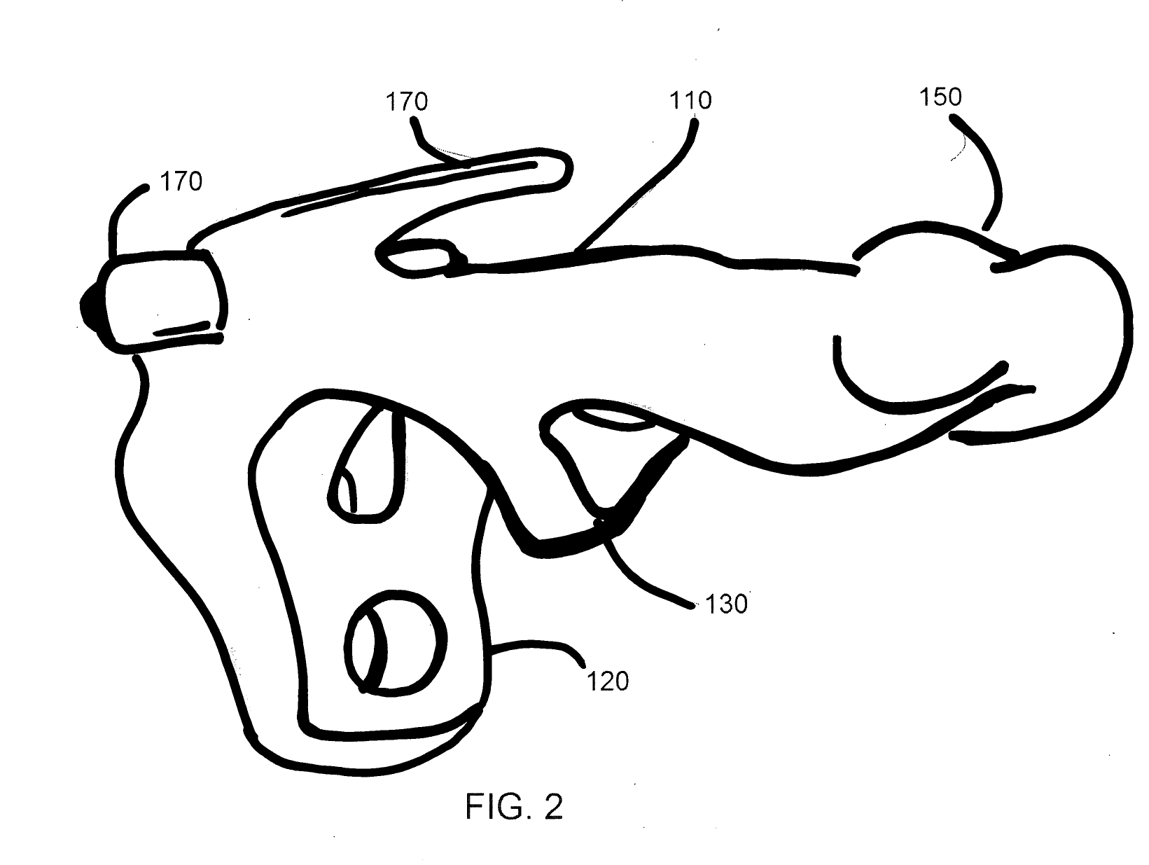

An erection enhancing device 100 for enhancing sexual stimulation for both a male and a female simultaneously or alone is described in FIGS. 1 through 3. The erection enhancing device 100 is an assembly comprising: a shaft base 110 oriented in a longitudinal direction that is conducive for supporting a penis 200. As referenced in FIG. 1, the shaft base 110 is configured to slidably accept the penis 200, and provide a framework structure for the erection enhancing device 100. In some embodiments, the shaft base 110 may comprise a soft skin tactile function. In alternative embodiments, the shaft base 110 may create a vacuum that further secures the penis 200 to the erection enhancing device 100. The erection enhancing device 100 further comprises a rear annular member 120 that attaches to the shaft base 110. The rear annular member 120 forms a perimeter around a penis shaft 210 and abuts against a lower abdomen 230 and a pair of testicles 240. The rear annular member 120 is often referred to as a “cock ring” in commercial embodiments, and is efficacious in restricting blood flow through the penis 200 and securing the penis 200 to the erection enhancing device 100. The erection enhancing device 100 similarly comprises at least one frontal annular member 130 attached to the shaft base 110 for securing the penis 200 to the erection enhancing device 100. The frontal annular member 130 forms a perimeter around the penis shaft 210 and is efficacious in restricting blood flow in the penis 200. The erection enhancing device 100 also has a longitudinal stiffening rod 140 located inside the shaft base 110. The longitudinal stiffening rod 140 is oriented longitudinally along the shaft base 110 for providing rigidity and width to the penis 200. Those skilled in the art can appreciate that the longitudinal stiffening rod 140 can be oriented in numerous positions and lengths to enhance sexual stimulation. The erection enhancing device 100 further comprises a bulbous distal member 150 attached to an extreme end of the shaft base 110 that abuts against the penis distal head 220 for providing length and width to the penis 200. Preferably, the bulbous distal member 150 is shaped to replicate a penis distal head 220. However, in other embodiments the bulbous distal member 150 can be configured in an assortment of shapes conducive to enhancing sexual stimulation. The erection enhancing device 100 further comprises a plurality of convex stimulators 160 attached to the shaft base 110, the rear annular member 120, the frontal annular member 130, and the bulbous distal member 150 (See FIG. 2). The convex stimulators 160 may include a penile gland tickler positioned inside the erection enhancing device 100 to engage the penis shaft 210 and provide sexual stimulation for the male.

The combination of the aforementioned components permits up to 80% of a penis surface area to remain uncovered and exposed; thereby enhancing the sexual stimulation for the male. The combination of the aforementioned components provide at least half an inch of elongation, increased width, and perpetual rigidity to the penis 200, thereby enhancing the sexual stimulation for the female and increasing the self-confidence for the male.

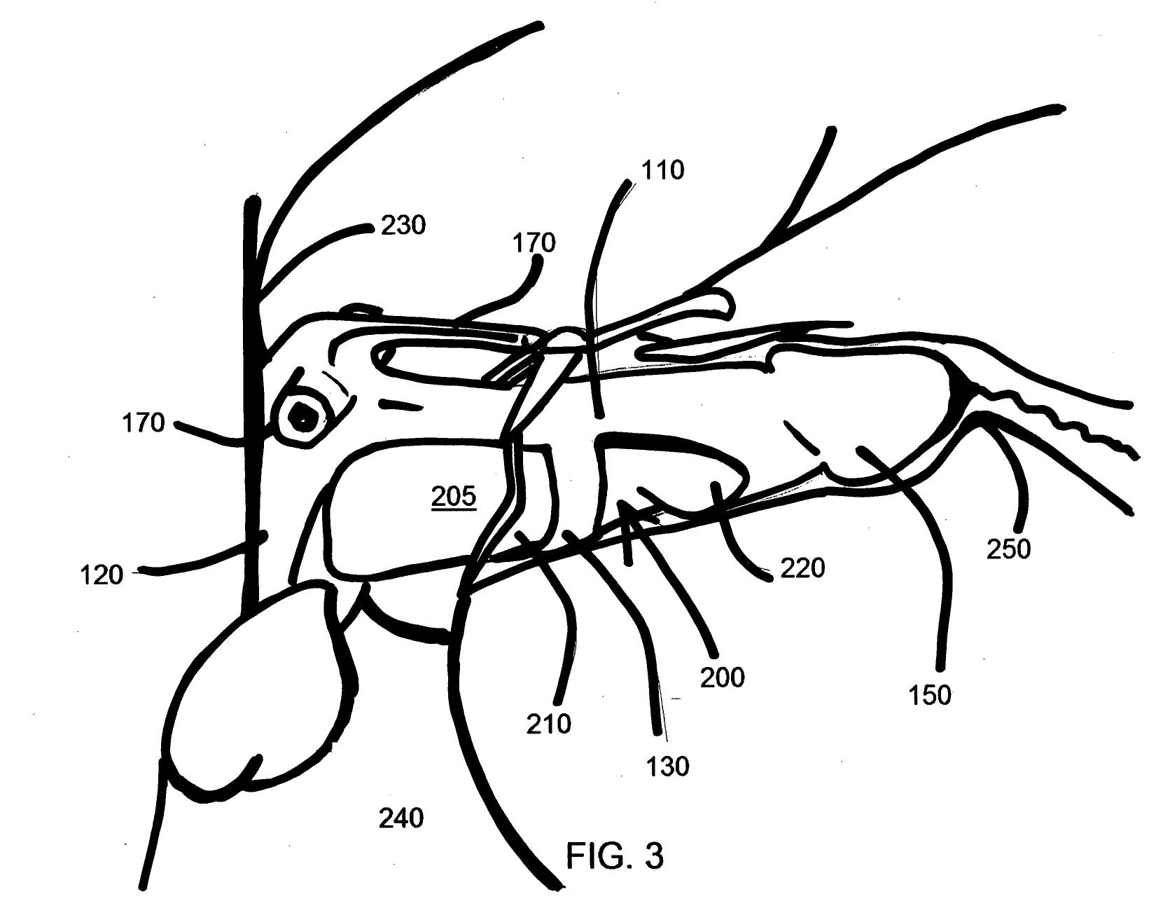

FIG. 3 illustrates one exemplary embodiment of the erection enhancing device 100 positioned onto the penis 200 and outside a vagina 250. The erection enhancing device 100 further comprises at least one vaginal stimulating member 170 for agitating the vagina 250. In some embodiments, the at least one vagina stimulating member 250 may include a clit tickler and a multi-speed vibrator. In some embodiments, the clit tickler is fabricated form a rigid material and positioned above the rear annular member 120 to provide additional resistance between the lower abdomen 230 and the vagina 250 during agitation and movement. The multi-speed vibrator may be positioned on the sidewall of the shaft base 110 for agitating opposing sides of the vagina. Those skilled in the art, in light of the present teachings, will recognize that the multi-speed vibrator may be positioned on the sidewall of the shaft base 110 for agitating opposing sides of the vagina. Those skilled in the art, in light of the present teachings, will recognize that the multi-speed vibrator may include a bulbous shape dimensioned to engage a large surface area of the interior of the vagina 250. Suitable materials for the multi-speed vibrator may include, without limitation, silicon, metal, plastic, and the like. In some embodiments, the multi-speed vibrators may include a portable power source that utilized a battery. In additional embodiments, the multi-speed vibrator utilizes three different speeds for varied stimulations. In one alternative embodiment, the vaginal stimulating member 170 allows for a female to utilize the erection enhancing device 100 alone, pushing the erection enhancing device 100 into the vagina 250 with hands.

Those skilled in the art can appreciate that in further embodiments, the erection enhancing device 100 can be sized to accommodate differently sized penises 200, regardless of whether the penises 200 are flaccid or rigid.

Those skilled in the art can appreciate that in further embodiments, the convex stimulators 160 can be shaped as beads, protruding ridges, a plurality of flexible members, internal penile gland ticklers, and the like for enhancing sexual stimulation.

Those skilled in the art can appreciate that in further embodiments, a vibrating device can attach to the erection enhancing device 100 for creating a vibrating motion that enhances sexual stimulation for both the male and female during use.

Those skilled in the art can appreciate that in further embodiments, an audio emitting device can attach to the erection enhancing device 10 for emitting music, advertising messages, or alarms during use.

Those skilled in the art can appreciate that in further embodiments, said erection emitting device 100 is edible.

Those skilled in the art can appreciate that in further embodiments, said vaginal stimulating member 170 can be a clit tickler and a multi-speed vibrator.

Those skilled in the art can appreciate that in further embodiments, said erection emitting device 100 can be flavored as fruit, chocolate, meat, and the like.

In operation, the erection enhancing device 100 is preferably utilized for a flaccid penis 200. However, a rigid penis 200 that is short or narrow could also benefit from the erection enhancing device 100. The first step for utilizing the erection enhancing device 100 requires examining the state of the penis 200. If the penis 200 is flaccid, the penis distal head 220 is pulled out into a 90 degree position. However, an aroused, normal penis 200 will automatically positioned to the 90 degree angle with minimal assistance. Next, the erection enhancing device 100 is oriented in front of the penis 200, so that the bulbous distal member 150 is farthest from the lower abdomen 230. The erection enhancing device 100 is then slidably pulled onto the penis 200 until the rear annular member 120 abuts against the lower abdomen 230 and the pair of testicles 240, and the bulbous distal member 150 abuts against the penis distal head 220. Despite the erection enhancing device 100 being fabricated in a manner that eliminates scratching or, harming the penis 200, the erection enhancing device 100 should still be gently pulled onto the penis 200. The penis 200, now rigid and extended, is then capable of positioning inside the vagina 250 for sexual activity. The female may utilize the erection enhancing device 100 alone because of the rigid structure and the at least one vagina stimulating member 170.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What I claim is:

1. An erection enhancing device for erecting and elongating a penis while providing significant exposure for a penis surface area and also stimulating a vagina, the erection enhancing device comprising:
a shaft base oriented in a longitudinal direction for supporting a penis, said shaft base configured to slidably receive said penis, said shaft base being further configured to allow up to 80% of the penis to remain exposed;
a rear annular member attached to said shaft base, said rear annular member forming a perimeter around a penis shaft and abutting against a lower abdomen and a pair of testicles, wherein said rear annular member restricts blood flow in said penis and secures said penis to said erection enhancing device;
a frontal annular member attached to said shaft base, said frontal annular member forming a perimeter around said penis shaft, wherein said frontal annular member restricts blood flow in said penis and secures said penis to said erection enhancing device;
a longitudinal stiffening rod located inside said shaft base, said longitudinal stiffening rod aligned longitudinally along said shaft base for providing rigidity and width to said penis shaft;
a bulbous distal member attached to an extreme end of said shaft base, said bulbous distal member abutting against a penis distal head, wherein said bulbous distal member provides length and width to said penis;
at least one convex stimulator attached to said erection enhancing device, wherein said convex stimulator is configured into a shape conducive for sexual stimulation; and
at least one vaginal stimulating member for agitating vagina.

2. The erection enhancing device of claim 1, wherein up to 80% of said penis surface area remains uncovered and exposed while utilizing said erection enhancing device.

3. The erection enhancing device of claim 1, wherein said erection enhancing device further includes at least one additional annular member.

4. The erection enhancing device of claim 1, wherein said bulbous distal member is shaped to replicate a penis distal head.

5. The erection enhancing device of claim 1, wherein said bulbous distal member is shaped to replicate a mushroom.

6. The erection enhancing device of claim 4, wherein said convex stimulator is attached to said rear annular member.

7. The erection enhancing device of claim 6, wherein said convex stimulator is attached to said frontal annular member.

8. The erection enhancing device of claim 7, wherein said convex stimulator is attached to said shaft base.

9. The erection enhancing device of claim 8, wherein said convex stimulator is shaped to resemble a bead.

10. The erection enhancing device of claim 8, wherein said convex stimulator is shaped to resemble a protruding ridge.

11. The erection enhancing device of claim 8, wherein said convex stimulator is shaped to resemble a plurality of flexible members.

12. The erection enhancing device of claim 1, wherein said at least one vaginal stimulating member is a multi-speed vibrator attached along the shaft base.

13. The erection enhancing device of claim 1, wherein said at least one vaginal stimulating member is a clit tickler attached to said rear annular member.

14. The erection enhancing device of claim 1, wherein a vibrating device having a portable power source attaches to said erection enhancing device, whereby said vibrating device causes said erection enhancing device to vibrate.

15. The erection enhancing device of claim 1, wherein an audio device having a portable power source emits sound attached to said erection enhancing device, wherein said audio device emits sound during sexual activity.

16. The erection enhancing device of claim 1, wherein said erection enhancing device is edible.

17. The erection enhancing device of claim 1, wherein said erection enhancing comprises a food flavor.

18. An erection enhancing device for erecting and elongating a penis while providing significant exposure for a penis surface area and also stimulating a vagina, the erection enhancing device comprising:
a shaft base oriented in a longitudinal direction for supporting a penis, said shaft base configured to slidably receive said penis;
a rear annular member attached to said shaft base, said rear annular member forming a perimeter around a penis shaft and abutting against a lower abdomen and a pair of testicles, wherein said rear annular member restricts blood flow in said penis and secures said penis to said erection enhancing device;
a frontal annular member attached to said shaft base, said frontal annular member forming a perimeter around said penis shaft, wherein said frontal annular member restricts blood flow in said penis and secures said penis to said erection enhancing device;
a longitudinal stiffening rod located inside said shaft base, said longitudinal stiffening rod aligned longitudinally along said shaft base for providing rigidity and width to said penis shaft;
a bulbous distal member attached to an extreme end of said shaft base, said bulbous distal member abutting against a penis distal head, wherein said bulbous distal member provides length and width to said penis; and
at least one convex stimulator attached to said erection enhancing device, wherein said convex stimulator is configured into a shape conducive for sexual stimulation.
